(19) United States
(12) Patent Application Publication
(10) Pub. No.: US 2013/0226107 A1
(43) Pub. Date: Aug. 29, 2013

(54) MEDICINAL ROLLER BALL APPLICATORS, ASSOCIATED PHARMACEUTICAL COMPOSITIONS, AND THEIR USE TO TREAT AFFLICTED SKIN TISSUES

(71) Applicant: Lexington Pharmaceuticals Laboratories, LLC, (US)

(72) Inventor: STEPHEN CRAIG FIELDS, Fishers, IN (US)

(73) Assignee: Lexington Pharmaceuticals Laboratories, LLC, Carmel, IN (US)

(21) Appl. No.: 13/778,601

(22) Filed: Feb. 27, 2013

Related U.S. Application Data
(60) Provisional application No. 61/634,275, filed on Feb. 27, 2012.

(51) Int. Cl. A61M 35/00

(52) U.S. Cl. CPC A61M 35/003
USPC 604/290

(57) ABSTRACT

The present invention discloses a novel medicinal roller ball applicator, comprising a ball with a rough surface, which can relatively painlessly facilitate the application and therapeutic massage of an active ingredient into a sensitive infected area of tissue for prolonged periods of time, while simultaneously removing dead epidermal tissue, a proven harrier to adsorption and penetration of active ingredients to the desired lower living skin tissue.

### FIELD OF THE INVENTION

The present invention relates to medicinal roller ball applicators, associated pharmaceutical compositions, and methods of using such applicators and compositions to treat afflicted skin tissues.

### BACKGROUND OF THE INVENTION

Significant medical research has focused on developing drugs, and formulations thereof, to treat afflicted skin tissues. Far less research has been directed to medical devices to deliver such drugs and formulations.

The outermost layer of the skin, the epidermis, is old dead skin. The new skin is being formed beneath it, in the innermost layer of the epidermis. It is recognized by those skilled in the art that the outer epidermis (i.e., stratum corneus) is a layer of dead skin cells Which do nothing but adsorb materials and is a major barrier to drug absorption. Use of transdermal-physical-medical devices to enhance penetration are techniques widely known to those of ordinary skilled in the art. However, they are either expensive (e.g., sonication and electrophoresis), need to be administered by a physician, or are unnecessarily invasive (e.g., microneedle technology Which causes either deep perforation of the epidermis or, at the very least, creation of grooves by dragging needles and/or spikes across the afflicted tissue prior to or during application of an active ingredient). In fact, there are warnings on one such commercial devices that under no circumstance should that technique be used on a cold sore for fear of irritating it and spreading the disease.

Examples of conventional compositions and application methods are provided In WO 98/42188 and WO 98/11778, which recite “spraying, dabbing, dusting, swabbing, sponging, brushing, pouring, dispensing, covering or heavily coating” a composition comprising Echinacea phytochemicals and benzalkonium chloride in a sterile water. The applied composition has a viscosity and/or tackiness wherein it substantially adheres to and remains on surface of the afflicted skin. Although a portion of the composition may eventually penetrate beyond the surface of the afflicted tissue, the viscosity of the composition and the application technique prevents such compositions from achieving the level of penetration required to administer an effective dose of the active ingredient.

U.S. Pat. No 5,753,270 discloses use of a cotton swab to apply an anti-herpetic composition. Cotton swabs are too abrasive to perform massage, as defined herein, without disruption of disordered tissue. Thus, the '270 patent describes application of active ingredients via frequent reapplication, specifically “liberal application 3 times in 10 minutes repeated for 3 hours” rather than extended massage of the active ingredient into the afflicted skin tissue.

U.S. Pat. No. 6,414,032 describes an anti-herpetic composition for treating cold sores in a skin disrupting system involving “vigorous agitation,” (also described as “therapeutic irritation”) to remove the skin and creating an open sore accompanied by “considerable pain” to the patient. More specifically, the method employs “vigorous agitation to the point of discomfort” and as such is preferably applied with a pain killer either before or after. The basic mechanism is that this “therapeutic irritation” creates an immune response that improves the healing process.

Medical devices and formulations for administering various medicinal formulations have been described to treat afflicted skin tissue such as, pumps, sprays, gauze, swabs, brushes, foams, creams and ointments. Creams and ointments typically require a finger to apply, which lacks the advantage of being clean or sterile, and the ability to finely abrade the skin surface to facilitate penetration of the active ingredient. In addition, application of sprays, pumps and foams are also often followed by spreading with a finger or not, in which case there is no massaging of the afflicted skin tissue. Moreover, while cotton swabs, gauze, brushes, and other abrasive materials could possibly be used to massage in the active ingredient, they generally significantly disrupt or cling to the afflicted skin tissue thereby causing discomfort and/or creating an open wound.

The commercial roller ball applicators designed to treat cold sores all have balls of machined smooth steel in order to evenly glide on either 100% homeopathic natural products like CITRUMED® and DOTERRA®, pain relievers like DR. SOLUTION®, emollients like aloe, or other materials like the sunscreens beeswax and zinc oxide. The instructions in the consumer packaging and/or websites indicate that these roller ball applicators are designed to facilitate complete coverage of the afflicted area, not prolonged massage of a medication in order to improve tissue penetration of the active ingredient.

It is widely acknowledged that dermal penetration of a medication enhances topical effectiveness, and that massaging in a medication increases dermal penetration. However, there is a need for a new way to effectively massage active ingredients into sensitive, afflicted skin tissue (1) in a sterile manner which (2) removes or uplifts the outermost skin surface, and (3) does not substantially remove, damage or irritate the underlying skin; (4) or result in substantial pain. The present invention addresses these problems via provision of a single device and thereby fulfills a long felt but unsolved need.

### SUMMARY OF THE INVENTION

There is no way for any of the aforementioned devices to massage in an active ingredient, which is an important aspect of the present invention, as it is widely known by those skilled in the art that massage facilitates penetration of chemical entities when properly formulated tar absorption. To be effective, an active ingredient must effectively penetrate the afflicted skin tissue, especially in the lip's dermal skin layer (e.g., cold sores where dead skin cells are abundant and constantly shedding).

The rough-surfaced roller ball of the present invention provides a unique combination of lifting dead skin cells to expose the live lower epidermal layer which is capable of both passive and active transport of active ingredients, while simultaneously facilitating relatively painless, lengthy massage of active ingredients into afflicted tissue.

The currently commercially available brushes and cotton swabs cannot be effectively used for massage as they are too abrasive and aggravate the sore by often removing the entire epidermis, causing exposure of the underlying sensitive dermis and resulting in considerable pain. Thus, the present invention invention, with the exception of gentle use of the human finger, is the only one that can claim to both lift, dead epithelial tissue and massage in the active ingredient with minimal pain. In addition, the inherent antiseptic advantage of the roller ball is obvious: it is a self cleansing applicator when using isopropanol, bleach, peroxide or any other anti-bacterial, anti-viral or anti-microbial medicinal chemicals such as benzalkonium chloride.

A first aspect of the present invention provides a novel medicinal roller ball applicator comprising a reservoir containing a pharmaceutical containing or more active ingredients selected from antivirals, antibiotics, antimicrobials, antifungals, anti-venoms and anesthetics wherein: the viscosity of the pharmaceutical composition is in a range which allows deliverability through a roller ball connected to a reservoir. Furthermore, this composition may also contain, in combination with any combination of active ingredients, a carrier, a surfactant, or other inert additives (e.g., with the intent to alter the smell, taste, color, texture or simply as a preservative).

A second aspect of the present invention provides methods for treating afflicted skin tissue by delivering an effective dose of an active ingredient into the tissue by massaging it with the medical roller ball applicator, which concurrently dispenses a pharmaceutical composition of one or more active ingredients selected from antivirals, antibiotics, antimicrobials, antifungals, anti-venoms and anesthetics wherein: the viscosity of the pharmaceutical composition is in a range which allows deliverability through a roller ball connected to a reservoir. Furthermore, this composition can optionally contain, in combination with the any combination of active ingredients, a carrier, an anesthetic, a surfactant, or other inert additives (e.g., with the intent to alter the smell, taste, color, or simply as a preservative).

It is a unique feature of present invention to allow simultaneously gently uplift and remove the top layer of dead lip skin cells, to prevent wasteful adsorption of the active ingredient onto non-living tissue, while concurrently and continuously applying medicine during massage of the active ingredient into the afflicted tissue with a single medical applicator.

Furthermore, the use of benzalkonium chloride as the active ingredient in isopropanol further reduces the number of ingredients, as the combination is an antiseptic/anti-viral/antimicrobial/antibiotic which guarantees the sterility of the roller ball and allows its repeated use without potential for cross-contamination.

### DETAILED DESCRIPTION OF THE INVENTION

The following definitions are used herein:

“Abraded ball” means the actual ball of the medicinal roller ball applicator which has slight surface imperfections and/or protuberances on its surface, used to create miniscule breaks in dead cell tissue and/or lift away dead skin cells to enhance penetration of active ingredients. This is synonymous with “rough-surfaced” and “non-smooth” roller ball. It is not meant from this definition that it itself is abrasive in the context of rolling the surface or that it creates any kind of “abrasion”. If it could not roll, then it certainly would.

“Abrade” means wear away, rub down, nib off, excoriate, chafe, or tear the skin.

“Abrasion” means an injury to living tissue (especially an injury in a cut or break in the skin).

“Active ingredient” (or AI) means a component of is pharmaceutical composition known to be biologically active, such as antivirals, antibiotics, antifungals, anti-venoms, anti-microbials and anesthetics.

“Applicator” means a medical device used to topically apply a pharmaceutical composition to afflicted skin tissue.

“Carrier” means the liquid and/or solution in which the active ingredient is dissolved, along with any other additives, in order to be delivered to afflicted skin tissue via the medicinal roller ball applicator. The carrier must have a viscosity/density consistent with application via roller ball. A topical application which offers external protection (via coating) to afflicted tissue, such as zinc oxide in sunscreen or beeswax, carmex, is not designed to penetrate tissue while carrying an active ingredient. Such topical applications are excluded from the inventions disclosed herein.

“Cold sore” means any skin surface disrupted/disorganized/infected localized skin disorder, commonly the result of the HSV-1 virus known as herpes.

“Effective amount” means the amount or dose of an active ingredient upon which single or multiple dose administration to a patient provides the desired treatment. It will be understood that physicians will generally determine the amount of active ingredient, or pharmaceutical composition thereof, actually administered under the relevant circumstances, including: the condition to be treated; the chosen route of administration; the physical properties and/or biological activities of the ingredient(s) or pharmaceutical formulation(s) themselves; the age, weight and general health of the patient; the response of the patient; and severity of the patient's condition.

“Massage” means using the roller ball to repeatedly roll a skin surface for any length of time (continually or not), in any direction, whether it be back and forth or in circles, under a pressure either advised by a physician or as guided by any pain from the applier, in order to facilitate penetration of the applied ingredient into the massaged tissue.

“Medicinal roller ball applicator” means a roller ball applicator used to apply a pharmaceutical composition. The device comprises a reservoir to hold and dispense a pharmaceutical composition connected (directly or indirectly) to a revolving ball fitted into the neck at one end of the container. As the ball is rolled over afflicted skin tissue, the pharmaceutical composition is dispensed from the reservoir and spread over afflicted tissue. Typically, the roller ball has a smooth surface. A “rough-surfaced” roller ball is described throughout the specification.

“Oral Herpes” means virus HSV-1 which creates afflicted tissue, such as cold sores.

“Patient” means a mammal, preferably a human.

“Penetration” means the passage of an active ingredient through and below the skin/dermal layer and/or tissue.

“Pharmaceutical composition” means a medicinal formulation comprising an active ingredient optionally combined with a carrier and/or additive, suitable to treat afflicted skin tissue.

“Quaternary ammonium compounds” have been shown to have antimicrobial activity^{[6]} Certain quaternary ammonium compounds, especially those containing long alkyl chains, are used as antimicrobials and disinfectants. Examples are benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride and domiphen bromide. These compounds are also good against fungi, amoeba, and enveloped viruses,^{[7]} since they act by disrupting the cell membrane. Quaternary ammonium compounds are lethal to a wide variety of organisms except endospores. Mycobacterium tuberculosis and non-enveloped viruses.

“Roller ball applicator” is a device comprises a reservoir to hold and dispense a composition connected (directly or indirectly) to a revolving ball fitted into the neck at one end of the device. As the ball is rolled over the skin, the composition is dispensed from the reservoir and spread over skin. Typically, the roller ball itself has a smooth surface. Roller ball applicators are well known in the art, e.g., for the delivery of cosmetics and deodorants/antiperspirants. They are intended to evenly spread a substance onto the surface of skin tissue which substantially remains on the surface. They do not create any significant miniscule breaks in skin tissue or remove surface skin cells.

“Skin Tissue” means epithelial and/or epidermal or dermal layers of the skin.

“Smooth roller ball applicator” means a roller ball applicator wherein the ball has a smooth surface.

“Rough-surfaced roller ball applicator” means a roller ball applicator wherein the ball has a non-smooth surface.

“Treatment” (or “treating” or “treat”) means mitigating, slowing, stopping, reducing, stopping the progression or severity of a symptom, disorder, condition or disease.

Medicinal Roller Ball Applicator

The first aspect of the present invention concerns a medicinal roller ball applicator. A medicinal roller ball applicator with a smooth ball is practical when using a pharmaceutical composition comprising a surfactant or other membrane penetrable carrier, or where an active ingredient is itself also a surfactant. Otherwise a non-smooth roller ball is preferred. The method of applying an active ingredient via massage is not to be confused with roller ball cosmetic applications, which are efficient at distributing the cosmetic for evenly and seamlessly, and wherein the Cosmetic substantially remains on the (generally normal) skin surface. In contrast, the medicinal roller ball applicator of the present invention facilitates a relatively painless massaging of a pharmaceutical composition into afflicted skin tissue with minimal disruption.

Conventional cosmetic roller ball applicators are also designed to apply a substantial amount of cosmetic over a relatively large area (e.g., the surface of the face) relative to the size of the roller ball. In contrast, the medicinal roller ball applicators of the present invention can have a roller ball approximately the same size as the skin tissue (e.g., cold sore) being treated. A pharmaceutical composition is typically applied to targeted tissue and perhaps the normal tissue a small distance around it. This results in less exposure of the patient to the pharmaceutical composition, thereby minimizing the potential for adverse side effects.

The disclosed medical roll on applicator invention has a reservoir, the contents of which come into contact with an application vehicle which is capable of rotation (e.g., a ball, wheel, cylinder or rocker). The applicator continually recoats the afflicted skin tissue as the active ingredient is massaged into the afflicted area over a period of anywhere from several seconds to several minutes using light to firm pressure. The material of the rolling surface can be anything from hard materials like metal, glass and plastic to soft materials such as cotton, felt, cloth, or any material in-between capable of being coated and delivering the contents of the reservoir in a controlled manner.

Reservoirs can be designed for either one-time treatment or for multiple treatments. They can also be disposable or reusable/refillable. Size and shape of the reservoir is not critical, although the preferred volume is less than 5 mL, such as between 1-2 mL, which would be adequate to treat a single infection. The reservoir can be either firm as in hard plastic or glass (such as in a perfume bottle) or malleable such as soft plastic or metal (such as a toothpaste tube) such that it can be squeezed to aid in dissemination of the contents of the reservoir via the roller ball.

The dimensions of the roiling entity are designed to treat a small area with massage, not large areas for coverage (like facial skin cosmetics or antiperspirants). Thus, a size of no greater than 1 cm is preferable all the way down to 1 mm. Delivery heads containing multiple roller balls are also consistent with this application as they could be designed to be small enough to cover a small area as well as lend themselves to massage.

Ultimately, the optimum size would be approximately no larger than the area to he covered, which in the case of a cold sore averages a diameter of 1 mm. This would prevent over application of medicine and possibly lessen any side effects from treatment of non-afflicted tissue. This minimizes the dose applied and the possible accompanying side effects. This contrasts with a pump or spray, where the amount of medicine dosed is uncontrolled and can exceed the amount necessary to control the skin malady as well as unnecessarily not only waste material but also coat healthy tissue creating the possibility of greater side effects from greater exposure.

The viscosity must be adequate to make the pharmaceutical composition flowable, which means the applicator must be able to effectively disseminate the contents of the reservoir via the roller ball. This will depend on the porosity of the roller ball itself as well as the distance of the gap between the roller ball and its housing. One could envision even a cream being applied successfully if the roller ball was coated with a cloth of some kind and there was sufficient gap between the roller ball and its housing. This would he tantamount to using a paint roller to apply a thin layer of paint to a wall and then being able to go over it repeatedly.

Alternatively, it is also an aspect of this invention that an externally applied medication (foam, gel, cream) could be massaged in subsequently by a secondary device capable of massaging, in the medication as described above.

A medicinal roller ball applicator has never been reported to apply a true non-holistic anti-herpetic medicine. In addition, a medicinal roller ball applicator has never been used to apply a tetra-alkonium halide to a cold sore. Further, a rough-surfaced roller ball applicator that can cause micro-perforations in the skin surface at the cellular level and/or lift and remove dead skin tissue without grossly and painfully abrading away the surface of the skin, enhances tissue penetration by an active ingredient, and renders use of surfactants less necessary, has never been described. These are accordingly aspects of the present invention.

Pharmaceutical Compositions for Use With Medicinal Roller Ball Applicator

The penetration into the afflicted skin tissue enabled by the disclosed gentle massaging application method using a medicinal roller ball applicator is further enhanced through the use of pharmaceutical compositions that have particular properties. These compositions include at least an active ingredient and a carrier, unless the active ingredient is can be applied without a carrier. The active ingredient is selected to effectively treat a specific skin tissue disorders (e.g., cold sores) and the carrier is selected to optimally enable the treatment composition to evenly flow from the medicinal roller ball applicator and to facilitate penetration into the afflicted skin tissue via gentle massage. The active ingredients, carriers and optional additives suitable for use in the pharmaceutical compositions of the present invention are set forth herein below.

An active ingredient as used herein means a component of a pharmaceutical composition known to be useful to treat a particular afflicted skin tissue. The term includes the following classes of drugs: antivirals, antibiotics, antifungals, and anesthetics and holistic remedies. Examples of suitable antivirals include Docosanol and other long chain fatty alcohols, or any medicinal chemicals from the family of quaternary ammonium halide salts such as benzalkonium chloride, acyclovir or any of the related cyclovir family of anti-herpetic drugs. Examples of suitable antibiotics include isopropanol, camphor, medicinal chemicals from the family of quaternary ammonium halide salts such as benzalkonium chloride. Examples of suitable antifungals include triazoles, imidazoles, polyenes, and allylamines. Examples of suitable anesthetics include painkillers such as lidocaine, xylocaine, benzocaine, phenol, menthol and the like. Examples of suitable holistic remedies include echinoa purpacae, Lysine, Vitamin E, aloe, sunflower oil, doTerra oils, chamomile, eucalyptus, tea tree and the like which are “all natural”.

Active ingredients for treatments of oral herpes cold sores include topical anesthetics such as lidocaine (found in DILOCAINE®, NERVOCAINE®, XYLOCANE®, and ZILACTIN-L®) to relieve pain and antibiotic and/or anti-viral topical active ingredients treatments of povidone-iodine, idoxuridine, trifluorothyidine, docosanol (found in ABREVA®) or benzalkonium chloride (found in VIROXYN® BACTICNE®, and RELEEV®) as well as acyclovir (found in ZOVIRAX®), Valacyclovir (found in VALTREX®), and famciclovir (found in FAMVIR®). There are also a number of holistic and/or natural product-based over the counter treatments such as Echinoa Purpur (wild violets) as the active ingredient.

Anti-infective agents included in the anti-infective treatment compositions are preferably anti-infective organohalides, especially anti-viral organohalides. Benzalkonium chloride is the preferred organohalide because it serves multi-purposes: it is proven to be anti-viral (including against HSV in vitro and in vivo), is antiseptic and is also a surfactant, thus replacing the role of three chemicals to do the job of one. While other organohalides or quatenary ammonium compounds have been tested in this faculty, benzalkonium chloride has been proven to be the most efficacious.

When the anti-infective agent is benzalkonium chloride, the concentration is preferably in a range from about 0.01% to about 0.5% by volume of the treatment composition, more preferably in a range from about 0.05% to about 0.3% by volume of the treatment composition, and even more preferably in a range from about 0.1% to about 0.2% by volume of the treatment composition. To avoid toxicity, the concentration is less than 0.26% and is most preferably about 0.13% by volume of the treatment composition. Depending on the particular, organohalide or quaternary ammonium chloride, the concentration may vary. For example, the concentration may range from about 0.001% to about 2% by volume of the treatment composition.

As indicated above, the carrier is a vehicle for the biologically active agent, more particularly the anti-infective active agent. The carrier causes effective wetting of the tissue to be treated and then enables the anti-infective agent to move within this carrier from the roller ball into the disordered tissue. The carrier has the viscosity to flow when the roller ball is rolled causing effective wetting of the tissue being treated and also enabling the active ingredient to move within this carrier into the afflicted skin tissue via roller ball massage.

The carrier has properties which facilitate the ability of the pharmaceutical composition to penetrate into the afflicted skin tissue. More particularly, the carrier has a viscosity and/or density which is about the same as water or lower in order to optimally enable the treatment composition to be applied via a roller ball applicator as well as penetrate into the afflicted skin tissue during massage. Using a carrier composition having a viscosity which is not greater than that of water is in sharp contrast to conventional compositions which enable the composition to be coated onto the surface of the afflicted skin tissue.

Accordingly, the pharmaceutical compositions of the present invention specifically exclude formulations which may be considered to be primarily or essentially gels, creams, lotions, oils, ointments, pastes, emulsions, and colloidal suspensions which do not have the proper flowability to be applied via roller ball. The carrier may have either a viscosity or density which is more than slightly greater than that of water as long as other substances are also included in the carrier such that the mixture has either a viscosity or density which is about the same as that of water or lower.

The carrier may be formed from a single liquid constituent such as water and/or an alcohol as described herein below, or from more than one constituent. Although water alone may be used as the carrier, it is generally not preferred because other compounds, such as some alcohols, have a tissue penetrating capability that water does not have. The carrier in the treatment composition is also preferably not formed entirely from an alcohol such as isopropyl alcohol or ethyl alcohol, since their use may be more painful in some circumstances. More particularly, when an open sore is part of the afflicted tissue, the amount of alcohol or other composition that has a significant tissue penetrating ability may be modified by adding water so as to moderate the amount of discomfort that the patient experiences by the application of the composition to the open sore.

The addition of water is also used to increase the viscosity in order to facilitate more effective roller ball applicator efficiency. When the carrier includes water and at least one alcohol, the water is preferably included in a range from about 10 percent to about 50 percent by volume of the carrier. The water content is more preferably in a range from about 20 to about 40 percent by volume of the carrier and is most preferably about 30 percent by volume of the carrier in order to optimize roller ball applicator efficiency. When other additives are included in the composition, it is essential that the amount of water and/or alcohol be adjusted to optimize roller ball efficiency.

The most preferred carrier is isopropyl alcohol as it is highly effective in penetrating tissue. While not being limited by any particularly theory, it is suggested that isopropyl alcohol opens cells and is not blocked by lipids or lipid layers in the disordered tissue. In addition to isopropyl alcohol, ethanol, and methanol are also suitable carriers. Benzyl alcohol is another preferred alcohol constituent as it also act as a bacteriostat and an anesthetic. Mixtures of the above-mentioned alcohols are also preferred depending upon the application. As indicated above, however, isopropyl alcohol or ethyl alcohol is preferably used in combination with other carrier constituents. For example, as mentioned above water may be added to isopropyl alcohol in order to reduce the pain which may be felt when only isopropyl alcohol is used. Similarly, isopropyl alcohol may be utilized with cetyl alcohol or with a combination of both cetyl alcohol and water to reduce the sensation.

It has also been noted that the ability of the treatment composition to penetrate disordered tissue is significantly enhanced when at least a portion of the carrier is an alcohol. It is believed that when at least a portion of the carrier is alcohol the carrier has the ability to remove lipids from the tissue.

When the carrier includes water and at least one alcohol, the water is preferably included in a range from about 10 percent to about 50 percent by volume of the carrier. The water content is more preferably in a range from about 20 to about 40 percent by volume of the carrier and is most preferably about 30 percent by volume of the carrier. Although, these ranges of water content are provided based on the volume of water in the carrier, essentially the same water contents apply to the overall treatment composition since the other active agent and any other optional component are typically included in such small amounts.

Other compounds that are preferred as carriers include acetic compounds such as acetone, acetic acid, acetic anhydride, and the like. While some acetic compounds may not be as effective as certain alcohols, particularly isopropyl alcohol, acetone exhibits an effective ability to penetrate tissue. One preferred carrier combination is ethanol and acetone in a ratio of about 70% ethanol by volume of the carrier, preferably 80% ethanol and most preferably about 90% ethanol with 10% acetone by volume of the carrier. As mentioned above with respect to the water content for carriers formed from water and alcohol, the ratios provided for combinations of ethanol and acetone apply also to the treatment composition.

The above carriers may also he combined across class lines. As such, the carriers such as water, alcohols, and acetic compounds may be combined. One example is water, alcohol, and acetone in respective amounts of 30%, 60%, and 10%, by volume of the carrier. Generally speaking, the constituents may be combined in an suitable ratio such as: 1:1:0, 1:2:0, 1:10:0, 1:1:1, 1:2:1, 1:10:1, 1:10:10, and 1:2:10.

As indicated above, the carrier preferably includes alcohol as it is believed that alcohols included in sufficient quantity to act as the carrier may have the quality of removing lipids from the tissue and thereby enabling the active agent to move within the disordered tissue. It is also believed that the ability of the treatment composition to penetrate the disordered tissue is hindered by including components in the composition such as oils or materials which have traditionally been included to enable the composition to be coated onto the surface of the disordered tissue.

The pharmaceutical compositions of the present invention can also include one of more additives, which herein mean an emollient, surfactant or any other inactive ingredient in a pharmaceutical composition.

### Embodiments of Medicinal Roller Ball Applicator

In a first embodiment, the medicinal roller ball applicator comprises a reservoir containing a pharmaceutical composition in contact with either a single or multiple revolving balls fined to an end of the reservoir.

In a second embodiment, the reservoir is in direct contact with the ball.

In a third embodiment, the reservoir is in indirect contact with the ball.

In a fourth embodiment, the roller ball device is designed for a single treatment.

In a fifth embodiment, the roller ball device is designed for multiple treatments.

In a sixth embodiment, the surface of the ball is smooth.

In a seventh embodiment, the surface of the ball is either rough-surfaced or has a non-smooth surface.

In an eighth embodiment, the surface of the ball has imperfections on its surface such that it is not smooth.

In a ninth embodiment, the ball is made of glass or porcelain.

In a tenth embodiment, the ball is made of metal or bard plastic.

In a eleventh embodiment, the imperfections on the surface of the ball surface are created by fabric, cotton, microfibers or any other materials that could alter the outer epidermal layer of the skin without abrasion if rolled, not scraped, across the surface.

In a twelfth embodiment, the surface of the ball has a coarse surface of the corresponding sandpaper grit of 60-1600.

In a thirteenth embodiment, the surface of the ball has a coarse surface of the corresponding sandpaper grit of 100-1200.

In a fourteenth embodiment, the surface of the ball has a coarse surface of the corresponding sandpaper grit of 400-1000.

In a fifteenth embodiment, the imperfections on the surface of the ball surface are of regular width, height and spacing.

In a sixteenth embodiment, the imperfections on the surface of the ball surface are of irregular regular width and height.

In a seventeenth embodiment, the imperfections on the surface of the ball surface are of regular square, or some other non-cylindrical or non-conical shape.

In a eighteenth embodiment, the diameter of the ball is ⅕ to 5 times the diameter of the disordered tissue (e.g., cold sore) to be treated.

In a nineteenth embodiment, the diameter of the ball is ⅓ to 3 times the diameter of the disordered tissue (e.g., cold sore) to be treated.

In a twentieth embodiment, the diameter of the ball is ½ to 2 times the diameter of the disordered tissue (e.g., cold sore) to be treated.

In a twenty-first embodiment, the reservoir is sealed with a fixed amount of the pharmaceutical composition.

In a twenty-second embodiment, the reservoir is refillable.

In a twenty-third embodiment, the reservoir is a squeezable tube.

In a twenty-fourth embodiment, the spacing between the ball and the end the end of the reservoir is matched to the viscosity of the pharmaceutical composition to facilitate easy and effective administration.

In a twenty-fifth embodiment, the pharmaceutical composition comprises one or more active ingredients selected from antivirals, antibiotics, antibacterials, antifungals, anesthetics, anti-microbials and antivenoms.

In a twenty-sixth embodiment, the antiviral is selected from topical treatments of povidone-iodine, idoxuridine, trifluorothyidine, docosanol (ABREVA), benzalkonium chloride (VIROXYN)Acyclovir (ZOVIRAX), Valacyclovir (VALTREX), and Famciclovir (FAMVIR). There are also a number of holistic and/or natural product-based over the counter (OTC) treatments such as Echinoa Purpur (wild violets, RELEEV) and L-lysine (HERPICIN) as the active ingredient.

In a twenty-seventh first embodiment, the active ingredient is an anesthetic.

In a twenty-eighth embodiment, the anesthetic is selected from lidocaine, benzocaine, nervocaine, or other known topically active agents known by those skilled in the art to be able to numb the skin surface or afflicted tissue.

In a twenty-ninth embodiment, the active ingredient is an antibiotic.

In a thirtieth embodiment, the antibiotic is selected from bactitracin, neomycin and others in the mycin family, iodines and iodate salts, hexidines, probiotics.

In a thirty-first embodiment, the pharmaceutical composition comprises one or more carriers selected from alcohols such as isopropanol, propylene glycols and derivatives, acetone, water, aqueous combinations of any of these, or any solvent or combination of solvents capable of creating the proper viscosity for application via a roller ball connected to a reservoir.

In a thirty-second embodiment, the carrier is aqueous isopropanol.

In a thirty-third embodiment, the pharmaceutical composition has a viscosity in the range of that of pure motor oil to that of diethyl ether.

In a thirty-fourth embodiment, the pharmaceutical composition has a viscosity in the range of that of pure isopropanol to that of water.

In a thirty-fifth embodiment, the pharmaceutical composition further comprises one or more additives.

In a thirty-sixth embodiment, the additive is selected from camphor, menthol, eucalyptol, peppermint oil, aloe, anethol, mineral oils, or sucrose stearates.

In a thirty-seventh first embodiment, the additive is an antiseptic.

In a thirty-eighth embodiment, the antiseptic is isopropyl alcohol, benzylalcol alcohol or other longer chain alcohols such as cetyl alcohol or docosanol and quaternary ammonium salts such as benzalkonium halides.

In a thirty-ninth embodiment, the additive is a surfactant.

In a fortieth embodiment, surfactant is a detergent.

In a forty-first embodiment, the active ingredient docosanol or benzalkonium chloride is its own surfactant.

In a forty-second embodiment, the carrier is also an antiseptic.

In a forty-third embodiment, the carrier is isopropanol.

In a forty-fourth embodiment, the carrier is aqueous isopropanol.

In a forty-fifth embodiment, the carrier is water.

In a forty-sixth embodiment, the anesthetic is lidocaine.

In a forty-seventh embodiment, the anesthetic is benzocaine.

In a funny-eighth embodiment, the additive is an inert substance designed to enhance the quality of the odor, texture, taste, flowability, or even be a simple preservative.

In a fourty-ninth embodiment, the inert additive is an alkyl paraben, a sorbate ester, a mineral oil, viracea, a perfume, any one of a number of emollients or perfumes.

Applicants emphasize that all logical, non-conflicting combinations of the above embodiments of the second aspect are contemplated and considered to he inventions within the scope of this application. All these combination are not specifically written out in order to minimize the length of the application itself.

Methods of Treatment Using the Medicinal Roller Bail Applicator

The second aspect of the present invention concerns methods of using the disclosed medical roller ball devices and pharmaceutical compositions to treat afflicted skin tissue.

Every inch of skin you see is old, dead skin. This is the outermost layer of the skin's outermost layer of they epidermis. The new skin is being formed beneath it, in the innermost layer of the epidermis. It is recognized by those skilled in the art that the outer epidermis (stratum cornetts) is the major barrier to drug absorption. Use of a transdermal physical device to enhance penetration is a technique known to those skilled in the art.

However, such techniques are either expensive (e.g., sonication or electrophoresis) and need to be administered by a physician, or are unnecessarily invasive such as patents such as the microneedle patents which describe deep perforation of the epidermis by needles and/or spikes prior to or during application of an active ingredient.

There is also no way any of these devices can be used to simultaneously massage in an active ingredient as it is being applied while lifting and removing dead skin cells Which each independently are widely known by those skilled in the art to facilitate penetration. This simultaneous trio of events is the key aspect of our invention.

As used herein, “massage” means application of a pharmaceutical composition across dermal tissue for a period of time particularly via use of a medicinal roller ball applicator, typically using typically gentle to moderate pressure. The amount of time (e.g., several seconds to several minutes) and pressure (e.g. gentle to moderate) to used is dependent on the concentration of the pharmaceutical composition, the abrasive nature of the roller ball, and the efficiency of the pharmaceutical composition in penetrating the afflicted skin tissue. Determination of such parameters is within the scope of one of ordinary skill in the art.

As an example which in no way is intended to limit the scope of the inventions enclosed herein, treatment can occur every few hours, if possible, with a minimum of twice per day for approximately 15-20 seconds per application. To reduce pain more effectively, apply a small amount first, and then wait 30 to 45 seconds to allow numbing aspect of the lidocaine to take effect. Then apply again and massage in.

Description of Afflicted Conditions That Can Be Treated

The medicinal roller ball applicator and associated pharmaceutical compositions of the present invention can be used to treat “afflicted skin tissue.” For purposes of this application “afflicted skin tissue” means infected (impure, tainted), disrupted (disturbed, upset), disordered (chaotic, tissue troubled, bothered, badly affected, distressed and/or problematic tissue. Included are cold sores, canker sores, fever blisters, burns, lesions, and other epithial (skin) disorder for which the pharmaceutical compositions of the invention is to be applied. Specific examples are disorders/infections created by Herpes, candida albicans, acne, psoriasis, eczema, seborrhea, dermatitis, and other bacterial or viral disorders in general, as well as microbial and fungal infections. Throughout this disclosure, use of the term “afflicted skin tissue” is understood to represent all tissue which has been affected by such disorders unless otherwise indicated.

Possible targets for use with this device are any topical disordered tissue which does or does not result from a systemic infection. Examples of systemic infections include any from the Herpes family (thus the shanker, cold sore or pox sores is that which is topically treated) or localized non-systemic bacterial infections like those found in acne and cuts and scrapes such as “turf burn”. Our preferred afflicted skin tissue is connected with the herpes virus. There are two common types of herpes: oral herpes simplex virus I (HSV-1, the most common form of oral herpes) and herpes simplex virus 2 (HSV-2, the most common form of genital herpes). The oral sores may occur on the lips, gums, front of the tongue, inside of the cheeks, throat, and roof of the mouth. In a preferred embodiment, the “afflicted skin tissue” being treated is the oral sores (e.g., cold sore, canker sores or fever blisters) associated with HSV-1.

### Embodiments of the Methods of Treatment

In a first embodiment, a method is provided for treating afflicted skin tissue by massaging, an effective dose of an active ingredient into the tissue with a medical roller ball applicator which concurrently dispenses a pharmaceutical composition comprising the active ingredient: wherein the applicator comprises a reservoir containing the pharmaceutical composition in contact with at least one revolving ball fitted to an end of the reservoir.

In a second embodiment, the reservoir is in direct contact with the ball.

In a third embodiment, the reservoir is in indirect contact with the ball.

In a fourth embodiment, the roller ball device is designed for a single treatment.

In a fifth embodiment, the roller ball device is designed for multiple treatments.

In a sixth embodiment, the surface of the ball is smooth.

In a seventh embodiment, the surface of the ball is rough or has a non-smooth surface.

In an eight embodiment, the surface of the ball has imperfections on its surface (i.e., is not smooth).

In a ninth embodiment, the ball is made of glass or porcelain.

In a tenth embodiment, the ball is made of metal or hard plastic.

In a eleventh embodiment, the imperfections on the surface of the ball surface are created by fabric, cotton, microfibers or any other materials that could alter the outer epidermal layer of the skin without abrasion if rolled, not scraped, across the surface.

In a twelfth embodiment, the surface of the ball has a coarse surface of the corresponding sandpaper grit of 60-1600. in a firth embodiment, the surface of the ball has a coarse surface of the corresponding sandpaper grit of 100-1200.

In a fourteenth embodiment, the surface of the ball has a coarse surface of the corresponding sandpaper grit of 400-1000.

In a fifteenth embodiment, the imperfections on the surface of the ball surface are of regular width, height and spacing.

In a sixteenth embodiment, the imperfections on the surface of the ball surface are of irregular regular width and height.

In a seventeenth embodiment, the imperfections on the surface of the ball surface are of a regular square or some other non-cylindrical or non-conical shape.

In an eighteenth embodiment, the applicator is used to treat afflicted skin tissue.

In an nineteenth embodiment, the afflicted skin tissue consists of isolated sores, lesions or ulcers, pox, or any other disordered tissue.

In a twentieth embodiment, the afflicted disordered skin tissue is in an area that may cause visible scarring, such as on the lips or around the face.

In a twenty-first embodiment, the disordered tissue may he of any shape.

In a twenty-second embodiment, the disordered tissue and or sores has been caused by a systemic disease such as Herpes I oral), II (genital), or VII (zoster).

In a twenty-third embodiment, the disordered tissue and/or sores has been caused by a localized bite, scrape or other injury prone to infection or already infected.

In a twenty-fourth embodiment, the size of the disordered tissue (e.g., isolated cold sores) is less than 1 inch in diameter.

In a twenty-fifth embodiment, the size of the disordered tissue (e.g., isolated cold sore) is less than ½ inch in diameter.

In a twenty-sixth embodiment, the afflicted tissue is a cold sore.

In a twenty-seventh embodiment, the diameter of the ball is ⅕ to 5 times the diameter of the disordered tissue (e.g., cold sore).

In a twenty-eighth embodiment, the diameter of the ball is ⅓ to 3 times the diameter of the disordered tissue (e.g., cold sore).

In a twenty-ninth embodiment, the diameter of the ball is ½ to 2 times the diameter of the disordered tissue (e.g., cold sore).

In a thirtieth embodiment, the reservoir is sealed with a fixed amount of the pharmaceutical composition.

In a thirty-first embodiment, the reservoir is refillable.

In a thirty-second embodiment, the reservoir is a squeezable tube.

In a thirty-third embodiment, the spacing between the ball and the end the end of the reservoir is matched to the viscosity of the pharmaceutical composition to facilitate easy and effective administration.

In a thirty-fourth embodiment, the pharmaceutical composition comprises one or more active ingredients selected from antivirals, antibiotics, antibacterials, antifungals. anesthetics, anti-microbials and antivenoms.

In a thirty-fifth embodiment, the antiviral is selected from topical treatments of povidone-iodine, idoxuridine, trifluorothyidine, docosanol (ABREVA), benzalkonium chloride (VIROXYN), Acyclovir (ZOVIRAX), Valacyclovir (VALTREX), and Famciclovir (FAMVIR). There are also a number of holistic and/or natural product-based over the counter (OTC) treatments such as Echinoa Purpur (wild violets, RELEEV), and L-lysine (HERPICIN) as the active ingredient AI).

In a thirty-sixth first embodiment, the active ingredient is an anesthetic.

In a thirty-seventh embodiment, the anesthetic is selected from lidocaine, benzocaine, nervocaine, or other known topically active agents known by those skilled the art to be able to numb the skin surface or afflicted tissue.

In a thirty-eighth embodiment, the active ingredient is an antibiotic.

In a thirty-ninth embodiment, the antibiotic is selected from bactitracin, neomycin and others in the mycin family, iodines and iodate salts, hexidines, probiotics.

In a fortieth embodiment, the pharmaceutical composition comprises one or more carriers selected from alcohols such as isopropanol, propylene glycols and derivatives, acetone, water, aqueous combinations of any of these, or any solvent or combination of solvents capable of creating the proper viscosity for application via a roller ball connected to a reservoir.

In a forty-first embodiment, the carrier is aqueous isopropanol.

In a forty-second embodiment, the pharmaceutical composition has a viscosity in the range of that of pure motor oil to that of diethyl ether.

In a forty-third embodiment, the pharmaceutical composition has a viscosity in the range of that of pure isopropanol to that of water.

In a forty-fourth embodiment, the pharmaceutical composition further comprises one or more additives.

In a forty-fifth embodiment, the additive is selected from camphor, menthol, eucalyptol, peppermint oil, aloe, anethol, mineral oils, or sucrose stearates.

In a forty-sixth first embodiment, the additive is an antiseptic.

In a forty-seventh embodiment, the antiseptic is isopropyl alcohol, benzyl a or other longer chain alcohols such as cetyl alcohol or docosanol and quaternary ammonium salts such as benzalkonium halides.

In a forty-eighth embodiment, the additive is a surfactant.

In a forty-ninth embodiment, surfactant is a detergent.

In a fiftieth embodiment, the active ingredient docosanol or benzalkonium chloride is its own surfactant.

In a fifty-first embodiment, the carrier is also an antiseptic.

In a fifty-second embodiment, the carrier is isopropanol.

In a fifty-third embodiment, the carrier is aqueous isopropanol.

In a fifty-fourth embodiment, the carrier is water.

In a fifty-fifth embodiment, the carrier enhances flowability.

In a fifty-sixth embodiment, the anesthetic is lidocaine.

In a fifty-seventh embodiment, the anesthetic is benzocaine.

In a fifty-eighth embodiment, the additive is an inert substance designed to enhance the quality of the odor, texture, taste, flowability, or even be a simple preservative.

In a fifty-ninth embodiment, the inert additive is an alkyl paraben, a sorbate ester, a mineral oil, viracea, a perfume, any one of a number of emollients or perfumes.

In a sixtieth embodiment, the coarse roller ball lifts dead skin cells to expose the live lower epidermal layer.

In a sixty-first embodiment, the coarse roller ball cause micro-perforations in the skin surface at the cellular level without grossly and painfully abrading away the surface of the skin.

In a sixty-second embodiment the roller ball could be used to gently apply the composition across extremely sensitive cold sore or afflicted tissue that has turned into an open sore yet.

In a sixty-third embodiment, the massage is performed with gentle to firm pressure.

In a sixty-fourth embodiment, the massage is performed over a period of several seconds to several minutes for each cold sore treated.

In a sixty-fifth embodiment, the massage is performed over a period of 10-30 seconds for each cold sore treated.

In a sixty-sixth embodiment, the treatment is performed on each cold sore a minimum of every few hours.

In a sixty-seventh embodiment, the treatment is performed on each cold sore a minimum of twice per day.

In a sixty-eighth embodiment, for pharmaceuticals compositions comprising an anesthetic, apply a small amount first then wait 30 to 45 seconds to allow numbing before massaging the pharmaceutical composition into a cold sore with the medicinal roller ball applicator.

Supporting Data

Applicant has compared smooth, stainless steel smooth roller ball applicators like those currently on the market with glass and plastic rough-surfaced roller balls of the present invention. When applying identical solutions of sufficient viscosity as described in this application, the solution in the vial with the metal roller ball remains clear while the one with our rough-surfaced roller ball becomes cloudy. This is evidence that very small amounts of dermal tissue are being delicately removed (and transferred into the vial containing the solution). Removal of this top layer of skin tissue correlates with better penetration.

Furthermore, in a limited human study (n=30), of those patients receiving treatment versus those with placebos, 75% of those receiving the medication described a decrease in the severity of cold sores versus only 30% of those receiving placebos.

1. A method for treating afflicted skin tissue b massaging an effective dose of an active ingredient into the tissue with a medical roller ball applicator which simultaneously dispenses a pharmaceutical composition comprising the active ingredient: wherein the applicator comprises a reservoir containing the pharmaceutical composition in contact with a revolving ball fitted to an end of the reservoir.

2. The method according of claim 1, wherein the surface of the bail is smooth.

3. The method of claim 1, wherein the surface of the ball is either rough or non-smooth.

4. The method of claim 3, wherein die rough or non-smooth surface is due to surface imperfections which alter the outer epidermal layer of the skin without creating an abrasion as the ball is rolled across the afflicted skin tissue during massage.

5. The method of claim 3, wherein the ball causes micro-perforations in the skin surface at the cellular level without grossly and painfully abrading, away the surface of the skin.

6. The method according to claim 3, wherein the ball lifts dead skin cells to expose the live lower epidermal layer.

7. The method of claim 3, wherein the rough or non-smooth surface has a coarseness corresponding to a sandpaper grit of 60-1600.

8. The method of claim 1, wherein the pharmaceutical composition comprises one or more active ingredients selected from: antivirals, antibiotics, antibacterials, antifungals, anesthetics, anti-microbials and antivenoms.

9. The method according to claim 8, wherein the active ingredient is an antiviral selected from: povidone-iodine, idoxuridine, trifluorothyidine, docosanol, benzalkonium chloride, acyclovir, valacyclovir, and famciclovir.

10. The method 8, wherein the active ingredient is an anesthetic selected from: benzocaine, butamben, dibucaine, lidocaine, oxybuprocaine, pramoxine, proparacaine, proxymetacaine, tetracaine and novocaine.

11. The method of claim 8, wherein the pharmaceutical composition comprises one or more carriers selected from isopropanol, ethylene or propylene glycol, acetone, and water.

12. The method of claim 1, wherein the afflicted skin tissue is disordered tissue selected from isolated sores, lesions, ulcers, pox, and acne.

13. The method of claims 12, wherein the disordered tissue has been caused by Herpes I (oral), II (genital), or VII (zoster).

14. The method of claim 12, wherein the disordered tissue is a cold sore.

15. The method of claim 14, wherein the massage is performed over a period of several seconds to several minutes for each cold sore treated.

16. The method of claim 12, wherein the disordered tissue is an extremely sensitive open sore.

17. The method according to claim 12, wherein the diameter of the hall is ⅕ to 5 times the diameter of the disordered tissue.

18. The method according to claim 1, wherein the pharmaceutical composition comprises benzalkonium chloride.
